(19) United States
(12) Patent Application Publication
(10) Pub. No.: US 2003/0014042 A1
(43) Pub. Date: Jan. 16, 2003

(54) Method of creating stromal pockets for corneal implants

(76) Inventors: Tibor Juhasz, Irvine, CA (US); Ronald M. Kurtz, Irvine, CA (US); J. Randy Alexander, Newport Beach, CA (US); Christopher Horvath, Irvine, CA (US); Carlos G. Suarez, Irvine, CA (US)

Correspondence Address: NEIL K. NYDEGGER, NYDEGGER & ASSOCIATES, 348 Olive Street, San Diego, CA, 92103, US

(21) Appl. No.: 09/905,635

(22) Filed: Jul. 13, 2001

(51) Int. Cl. A61B 18/18; A61F 2/14; A61F 9/00

(52) U.S. Cl. 606/5; 606/166; 623/5.11; 623/5.12

(57) ABSTRACT

A surgical method for vision correction includes the step of first determining the type, size and shape of a corneal implant and a location within the stroma for placement of the corneal implant. Next, the dimensions of a stromal pocket suitable for accommodating the corneal implant are prescribed. To create the stromal pocket, a pulsed laser beam is focused to a point within the stromal tissue and then moved within the stromal tissue to photodisrupt the prescribed volume of stromal tissue. Once the stromal pocket is established, an entry channel extending from the anterior surface of the eye to the stromal pocket is created. The entry channel is sized to allow the prescribed corneal implant to pass through the entry channel and into the stromal pocket.

### FIELD OF THE INVENTION

The present invention pertains generally to ophthalmic surgery which is useful for correcting vision deficiencies. More particularly, the present invention pertains to methods which surgically alter the refractive properties of the cornea to correct the vision of a patient. The present invention is particularly, but not exclusively useful as a method for correcting the vision of a patient by creating a stromal pocket and an entry channel to the pocket with a laser beam, and subsequently passing a biocompatible corneal implant through the entry channel and into the stromal pocket.

### BACKGROUND OF THE INVENTION

Vision impairments such as myopia (i.e. near-sightedness), hyperopia (i.e. farsightedness) and astigmatism can be corrected using eyeglasses or contact lenses. Alternatively, the cornea of the eye can be reshaped surgically to provide the needed optical correction. For example, it is known that if part of the corneal stroma is removed, the pressure exerted on the cornea by the aqueous humor in the anterior chamber of the eye will act to close the created void. The result is a reshaped cornea. One technique which relies on the formation of a void within the stroma to reshape the cornea is the LASIK (laser in situ keratomileusis) procedure. Unfortunately, traditional LASIK procedures generally use a microkeratome to create the flap, which may cause excessive tissue damage requiring an undesirably lengthy healing period. As an example of another such procedure, U.S. Pat. No. 5,993,438 which issued to Juhasz et al. for an invention entitled “Intrastromal Photorefractive Keratectomy,” discloses an intrastromal photodisruption technique for reshaping the cornea. Importantly for the purposes of the present invention, the above cited Juhasz patent discloses the use of a non-ultraviolet, ultrashort, pulsed laser beam having pulses with durations measured in femtoseconds for photodisruption of intrastromal tissue. As disclosed, the pulsed laser beam propagates through corneal tissue and is focused at a point below the surface of the cornea to photodisrupt stromal tissue at the focal point. Importantly, photodisruption does not depend on absorption of laser energy by the tissue. Rather, laser energy is concentrated in time by ultrashort pulse durations and in space by extremely small spot sizes resulting in the creation of very high electric fields that induce a process termed optical breakdown and plasma formation. The ability to reach a subsurface location without necessarily providing a physical pathway allows for the creation of stromal voids or pockets having complex shapes while minimizing the total amount of tissue disrupted.

Another known technique commonly used to correct the vision of a patient involves inserting a corneal implant within the stroma of the cornea. In this technique, a stromal pocket is created by either making an incision in the stromal layer of the cornea, or by removing a small amount of stromal tissue. Next, a corneal implant is placed in the stromal pocket to either reshape the cornea, alter the refractive properties of the cornea, or both. Unlike the LASIK procedure, one advantage of using a corneal implant is that the effects of the procedure can often be reversed by the subsequent removal of the corneal implant.

Heretofore, stromal pockets have been prepared to accommodate lens and disk-shaped corneal implants by mechanically incising and separating the corneal lamellae. Specifically, a slit knife is used to create an incision from the anterior surface of the cornea to the desired depth of the implant. Next, a flat blunt blade is used to separate the corneal lamellae over the central optical zone of the cornea, thereby creating a stromal pocket. For the purposes of the present disclosure, the central optical zone of the cornea is defined as the portion of the cornea that substantially overlies the pupil and is surrounded by the corneal periphery.

Another technique, known to be effective for corneal reshaping involves inserting a corneal ring within the stromal tissue of the cornea. One of the advantages of using ring-shaped implants is that myopia can be corrected without destroying corneal tissue in the central optical zone of the cornea. Specifically, a corneal ring consisting of either a single continuous annular ring, two half-rings, a ring segment or a pair of crescent shaped corneal implants can be embedded within the stromal tissue that surrounds the central optical zone of the cornea (i.e. the corneal periphery) to flatten the curvature of the cornea. In this technique, a stromal pocket in the shape of an annular channel is established in the corneal periphery and an access slot is created extending from the annular stromal pocket to the anterior surface of the cornea. Next, a ring-shaped corneal implant is passed through the access slot and into the annular stromal pocket, thereby reshaping the central optical zone of the cornea.

Heretofore, the preparation of an annular stromal pocket to accommodate a ring-shaped corneal implant has been performed by first making an incision extending from the anterior surface of the cornea to the intended depth of the implant. Next, a mechanical spatula that resembles a thin, flat corkscrew is inserted into the incision and turned to separate the corneal lamellae. The spatula is removed from the cornea, leaving an annular shaped channel in the stromal tissue.

Unfortunately, the mechanical procedures outlined above rely on the skill of the surgeon and may subject the eye to grossly elevated intraocular pressures and mechanical trauma. Additionally, the blunt blade incision typically results in a pocket that either is not geometrically in a single stromal plane, or is in a plane that is not perpendicular to the corneal optical axis, or both. When an implant is inserted into such an incision, induced astigmatism results. Moreover, it is known that the effectiveness of corneal rings depends on the depth of the channel incision. However, mechanical means of creating such incisions do not precisely control the final depth of the rings. Further, creation of a stromal pocket by mechanical means may result in infection, corneal edema and corneal tearing. Finally, the mechanical procedures are labor intensive and often lack the accuracy needed to create an adequate stromal pocket.

In light of the above, it is an object of the present invention to provide a method for establishing a stromal pocket suitable for accommodating an optical or biomechanical corneal implant that minimizes trauma to the corneal tissue, and the risks of elevated intraocular pressure and infection. Another object of the present invention is to provide a method for corneal laser surgery which allows for a precisely shaped stromal pocket to be established in the cornea. Another object of the present invention is to provide a method by which the depth of the implant within the corneal tissue is precisely controlled. Yet another object of the present invention is to provide a method for corneal laser surgery which is relatively easy to practice and comparatively cost effective.

### SUMMARY OF THE PREFERRED EMBODIMENTS

In accordance with the present invention, a method for corneal laser surgery includes the step of first prescribing the type and dimensions of a corneal implant. The corneal implant may be an optical lens for altering the refractive properties of the cornea or a biomechanical implant for altering the shape of the cornea. Once the size, type and shape of the implant are selected, the location within the cornea where placement of the implant will effectively correct the vision deficiency of a patient is prescribed. In accordance with the present invention, the implant location will preferably be entirely within the stroma of the cornea. Next, the dimensions of a stromal pocket suitable for receiving and containing the corneal implant are determined. The size of the stromal pocket may take into account the fact that some corneal implants may swell after implantation due to hydration.

To create the stromal pocket, first the exterior surfaces of the stromal pocket are identified. In general, a suitable stromal pocket for accommodating a corneal implant is defined by an anterior surface, a posterior surface and a peripheral edge connecting the anterior surface to the posterior surface. The size of the peripheral edge defines the thickness of the stromal pocket.

For the present invention, a pulsed laser beam is focused to a preselected start point within the stromal tissue. In accordance with preplanned procedures, the focal point is preferably located on the posterior surface of the prescribed stromal pocket. The focal point is then moved within the stromal tissue to cut (photodisrupt) a layer of tissue having the desired contour of the posterior surface of the prescribed stromal pocket that is being created. This photodisrupted layer of tissue has a thickness approximately equal to the focal point diameter of the pulsed laser beam. In many applications, a thin stromal pocket is prescribed requiring only a single layer of stromal tissue to be photodisrupted. When a thicker stromal pocket is prescribed, after photodisruption of the prescribed posterior surface, the focal point can be moved within the stroma to photodisrupt the volume of stromal tissue that lies between the prescribed posterior surface and the prescribed anterior surface.

Once the stromal pocket has been established, an entry channel is created that is sized to allow the prescribed corneal implant to pass from an extracorporeal location, through the entry channel and into the stromal pocket. To create the entry channel, an incision is made in the cornea extending from the anterior surface of the cornea to the stromal pocket. Preferably, the incision is made using a pulsed laser beam. It is to be appreciated that other techniques known in the pertinent art can be used to establish the entry channel such as making the incision with a surgical knife. Once the entry channel is established, gas bubbles and debris can be evacuated from the stromal pocket using techniques well known in the pertinent art. Next, the corneal implant can be passed through the entry channel and positioned in the stromal pocket.

In another embodiment of the present invention, a stromal pocket suitably shaped to receive an implant can be created by excising a volume of stromal tissue from the cornea. In this embodiment, a pulsed laser beam is used to detach a volume of stromal tissue from the stroma for subsequent excising. Specifically, a pulsed laser beam is focused to a pre-selected start point within the stromal tissue. In accordance with preplanned procedures, the focal point is preferably located on the posterior surface of the prescribed stromal pocket. The focal point is then moved within the stromal tissue to cut (photodisrupt) a layer of tissue having the desired contour of the posterior surface of the prescribed stromal pocket that is being created. Next, the focal point is moved within the stroma to photodisrupt the peripheral edge and anterior surface of the pocket, leaving a volume of stromal tissue detached in the stroma. For ease of removal, the volume of stromal tissue may be sectioned using the pulsed laser beam. After detachment of the volume of stromal tissue, an entry channel can be incised extending from the anterior surface of the cornea to the detached volume. The entry channel can then be used to excise the volume of stromal tissue thereby establishing the prescribed stromal pocket. Next, the corneal implant can be passed through the entry channel and positioned within the stromal pocket.

It should be appreciated that in all of the above-mentioned procedures, the effectiveness of the implant depends in some measure to the depth within the stroma where the implant is received. In the case of the present invention, cutting (photodisruption) is achieved only at the focus point of the pulsed laser. Since the focus point of the laser is precisely controlled (preferably by means of a fast computer control), it follows that the depth of the incision is similarly controlled.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a patient being treated with a pulsed laser in accordance with the method of the present invention;

FIG. 2 is a perspective view of an eye;

FIG. 3 is a cross sectional view of a portion of the cornea of the eye as seen along the line 3-3 in FIG. 2 showing the anatomical layers of the cornea;

FIG. 4A is a perspective view of a disk-shaped corneal implant;

FIG. 4B is a perspective view of a ring-shaped corneal implant;

FIG. 4C is a sectional view through a lens-shaped corneal implant;

FIG. 5 is a plan view of the cornea as seen along line 5-5 in FIG. 2 showing the cornea after the photodisruption of a stromal pocket (shown in phantom) and the incision of an entry channel (shown in phantom);

FIG. 6 is a cross-sectional view of a cornea as seen along the line 6-6 in FIG. 5, showing a stromal pocket and entry channel;

FIG. 7 is a plan view of the cornea as in FIG. 5 showing the cornea after a disk-shaped volume of stromal tissue (shown in phantom) has been detached by photodisruption and an entry channel (shown in phantom) has been incised;

FIG. 8 is a cross-sectional view of a cornea as seen along the line 8-8 in FIG. 7, showing a detached volume of stromal tissue and an entry channel;

FIG. 9 is a cross-sectional view of a cornea as seen in FIG. 8 showing a reshaped cornea that results after implantation of a corneal implant (solid lines), superimposed over a cornea prepared with a stromal pocket (dotted lines);

FIG. 10 is a plan view of the cornea as seen along line 10-10 in FIG. 2 showing the cornea after the photodisruption of a channel-shaped stromal pocket (shown in phantom) and the incision of an entry channel (shown in phantom); and

FIG. 11 is a cross-sectional view of a cornea as seen along the line 11-11 in FIG. 10, showing a channel-shaped stromal pocket and an entry channel.

### DESCRIPTION OF THE PREFERRED EMBODIMENT

Referring initially to FIG. 1, an apparatus 13 for generating a pulsed laser beam 14, focusing the pulsed laser beam 14 and moving the focal point of the pulsed laser beam 14 is shown. In detail, FIG. 1 shows the pulsed laser beam 14 being directed onto the eye 15 of a patient 16. For purposes of the present invention, a non-ultraviolet, ultrashort, pulsed laser beam 14 is preferably used. Furthermore, a pulsed laser beam 14 having pulses with durations as long as a few nanoseconds or as short as only a few femtoseconds is preferably used for the present invention. More preferably, a pulsed laser beam 14 having pulse durations between approximately 500 picoseconds and 10 femtoseconds, and a wavelength longer than approximately 800 nanometers is used. Also, the pulsed laser beam 14 preferably has a fluence of less than 100 joules per square centimeter. In accordance with the present invention, a software program is preferably created containing the instructions for controlling the position and movement of the focal point to accomplish the methods of the present invention. Preferably, the software program is stored on a computer readable medium for use during the procedure by a computer processor in the apparatus 13.

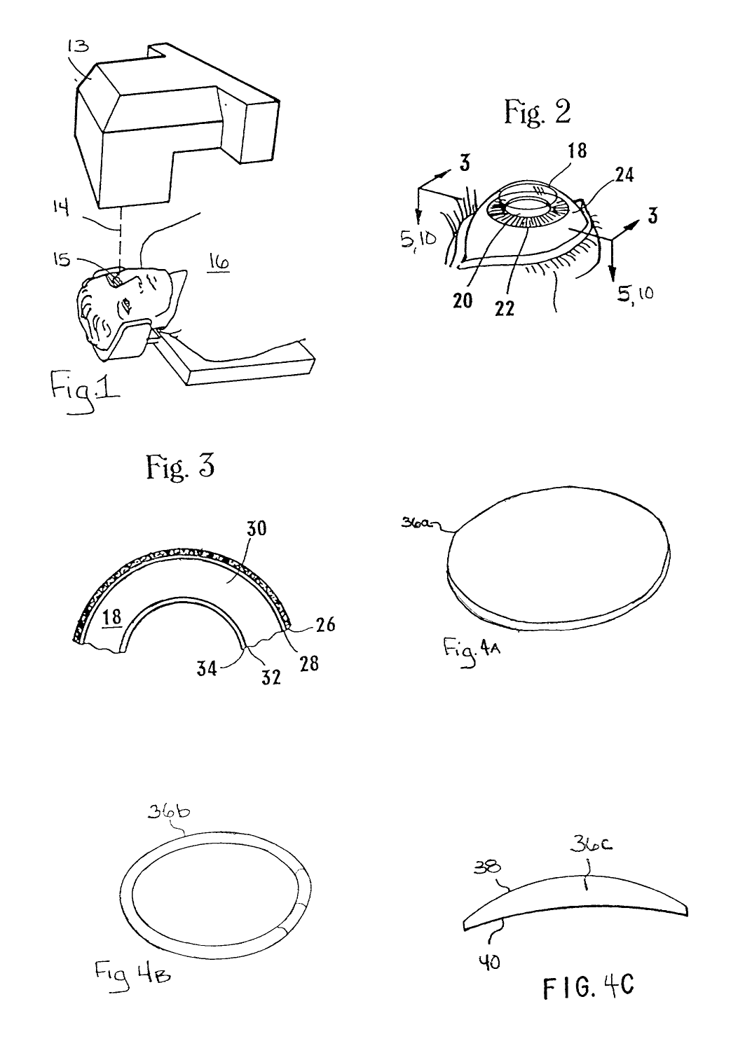

FIG. 2 shows the anatomical structure of the human eye 15 including the cornea 18, the pupil 20, the iris 22, and the sclera 24. In FIG. 3 it can be seen that the cornea 18 includes five anatomically definable layers of tissue. Going in a direction from anterior to posterior in FIG. 3, the tissue layers of the cornea 18 are: the epithelium 26, Bowman's membrane 28, the stroma 30, Decemet's membrane 32 and the endothelium 34. Of these, the stroma 30 is of most importance for the present invention as it contains the stromal tissue which is to be removed to allow a corneal implant to be positioned in the cornea 18.

Exemplary corneal implants 36 for use with the present invention are shown in FIGS. 4A-C. As shown, the corneal implant 36 can be a disk-shaped implant 36a or a ring-shaped implant 36b to correct a vision deficiency by altering the shape of the cornea 18, or the corneal implant 36 can be a lens-shaped implant 36c to correct a vision deficiency by altering the refractive properties of the cornea 18. For purposes of the present invention, a ring-shaped implant 36b similar to the rings as disclosed in U.S. Pat. No. 5,888,243 entitled “Hybrid Intrastromal Corneal Ring” and U.S. Pat. No. 5,824,086 entitled “Segmented Pre-Formed Intrastromal Corneal Insert,” which issued to Silvestrini, may be used. For the present invention, the ring-shaped implant 36b may be a single continuous annular ring having a square or round cross section. Alternatively, the ring-shaped implant 36b can consist of two half-rings, a ring segment, a pair of crescent shaped corneal implants or any other substantially ring-shaped implant known in the pertinent art. For the present invention, the disk-shaped and ring-shaped implants 36a,b may be fabricated from a clear, medical grade plastic, polymeric materials, cellulose esters, hydrogel materials, silicone, bio-engineered tissue, or other biocompatible materials.

For purposes of the present invention, a lens-shaped implant 36c similar to the inlay lenses disclosed in U.S. Pat. No. 5,336,261 entitled “Corneal Inlay Lenses,” which issued to Barrett et al., may be used. For the present invention, both positive and negative lenses of all useful diopters may be employed. Further, the lenses may be of a refractive index greater than, less than, or equal to that of the neighboring corneal tissue. For the present invention, the first spherical surface 38 of the lens-shaped implant 36c may be concave, convex or planar. Similarly, the second spherical surface 40 of the lens-shaped implant 36c may be concave, convex or planar. Further, it is to be appreciated that in accordance with the present invention, a lens-shaped implant 36c can be inserted into the stroma 30 to correct a vision deficiency by altering both the refractive properties of the cornea 18 as well as altering the shape of the cornea 18.

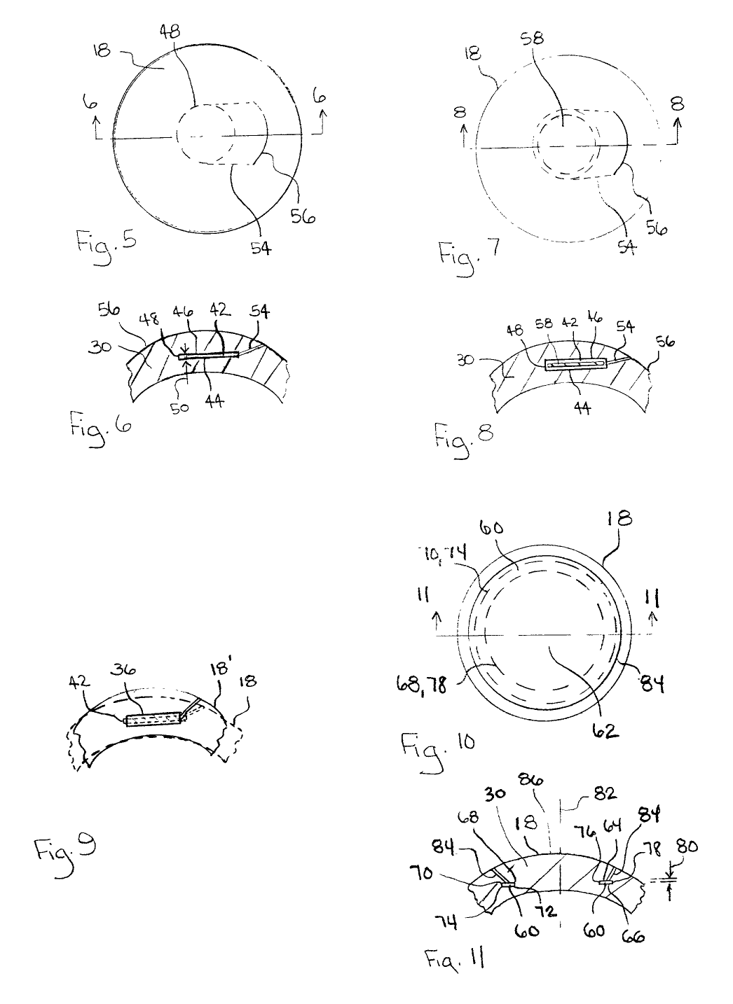

Referring to FIGS. 5 and 6, in accordance with the methods of the present invention, a stromal pocket 42 is established in the stroma 30. For the present invention, the stromal pocket 42 can be located entirely within the central optical zone of the cornea, entirely within the corneal periphery, or partially in both the central optical zone and the corneal periphery. It is to be appreciated that the stromal pocket 42 is sized and located to accommodate a corneal implant 36 within the stromal pocket 42. Although FIGS. 5 and 6 show a stromal pocket 42 positioned in the central optical zone of the cornea 18 for accommodating a disk-shaped biomechanical implant 36, it is to be appreciated that the stromal pocket 42 can be positioned anywhere within the stroma 30 of the cornea 18. As shown in FIG. 6, the stromal pocket 42 can be formed with a posterior surface 44, an anterior surface 46 and a peripheral edge 48 that connects the posterior surface 44 to the anterior surface 46. Preferably, the anterior surface 46 and the posterior surface 44 of the stromal pocket 42 are shaped to conform to the surfaces of the corneal implant 36. As such, the surfaces 44, 46 can be convex, concave, planar or irregular. As shown in FIG. 6, the posterior surface 44 is generally separated from the anterior surface 46 by a thickness 50.

For the present invention, the pulsed laser beam 14 is focused to a pre-selected start point within the stromal tissue 30. It is contemplated for the present invention that the focal point diameter of the pulsed laser beam 14 will be between approximately 1 μm and approximately 100 μm. In accordance with preplanned procedures, the focal point is preferably located on the posterior surface 44 of the prescribed stromal pocket 42. The focal point is then moved within the stromal tissue 30 to cut (photodisrupt) a layer of stromal tissue 30 having the desired contour of the posterior surface 44 of the prescribed stromal pocket 42 that is being created. This photodisrupted layer of tissue has a thickness approximately equal to the focal point diameter of the pulsed laser beam 14. In many applications, a stromal pocket 42 is prescribed having a thickness 50 that is approximately the size of the focal point diameter of the pulsed laser beam 14 (i.e. a thin stromal pocket 42 is prescribed). In these applications, only photodisruption of a single layer of stromal tissue 30 is required to create the stromal pocket 42. In other applications, the prescribed thickness 50 for the stromal pocket 42 may exceed the size of the focal point diameter of the pulsed laser beam 14 (i.e. a thick stromal pocket 42 is prescribed). For these applications, further stromal photodisruption can be performed after photodisruption of the prescribed posterior surface 44. Specifically, the focal point of the pulsed laser beam 14 can be moved within the stroma 30 to photodisrupt the volume of stromal tissue that lies between the prescribed posterior surface 44 and the prescribed anterior surface 46, and is bounded by the peripheral edge 48, thereby creating the stromal pocket 42. In this manner various sizes and shapes of stromal pockets 42 can be created.

In accordance with the methods of the present invention, an entry channel 54 is created that is sized to allow the prescribed corneal implant 36 to pass from an extracorporeal location, through the entry channel 54 and into the stromal pocket 42. As can be seen by cross-referencing FIGS. 5 and 6, an incision is made in the cornea 18 extending from the anterior surface 56 of the cornea 18 to the stromal pocket 42 to create the entry channel 54. Preferably, the incision is made using a pulsed laser beam 14, thereby minimizing the amount of tissue disruption and allowing for an accurate incision. It is to be appreciated that other techniques known in the pertinent art can be used to establish the entry channel 54 such as making the incision with a surgical knife. Once the entry channel 54 is established, gas bubbles and debris resulting from the photodisruption of tissue can be evacuated, if desired, from the stromal pocket 42 using techniques well known in the pertinent art. For example, a suction pump (not shown) can be positioned over a portion of the anterior surface 56 of the cornea 18 and in fluid communication with the entry channel 54. Subsequently, the suction pump can be activated to aspirate any gas and debris from the stromal pocket 42, through the entry channel 54 and out of the cornea 18. Alternatively, the entry channel 54 can be opened with an ordinary surgical tool to allow the gas to escape and any debris to be removed. Once the entry channel 54 has been created, the corneal implant 36 can be passed through the entry channel 54 and positioned in the stromal pocket 42.

Referring now to FIGS. 7 and 8, in another embodiment of the present invention, the stromal pocket 42 can be created by excising a volume of stromal tissue 58 from the cornea 18 leaving a stromal pocket 42 having a suitable shape for receiving and containing a corneal implant 36. In this embodiment, the pulsed laser beam 14 is used to detach the volume of stromal tissue 58 from the remaining stromal tissue 30 prior to excising. Specifically, in accordance with the methods of the present invention, a pulsed laser beam 14 is focused to a pre-selected start point within the stromal tissue 30. In accordance with preplanned procedures, the focal point is preferably located on the posterior surface 44 of the prescribed stromal pocket 42. The focal point is then moved within the stromal tissue 30 to cut (photodisrupt) a layer of stromal tissue 30 having the desired contour of the posterior surface 44 of the prescribed stromal pocket 42 that is being created. Next, the focal point is moved within the stroma 30 to photodisrupt the peripheral edge 48 and anterior surface 46 of the stromal pocket 42, leaving the volume of stromal tissue 58 detached in the stroma 30. For ease of removal, the volume of stromal tissue 58 may be further sectioned using the pulsed laser beam 14.

After detachment of the volume of stromal tissue 58, an entry channel 54 can be incised extending from the anterior surface 46 of the cornea 18 to the detached volume of stromal tissue 58. The entry channel 54 can then be used as a pathway to excise the volume of stromal tissue 58 thereby establishing a stromal pocket 42. Also, the entry channel 54 can be used to remove any gas bubbles or debris resulting from the photodisruption operation. Finally, the corneal implant 36 can be passed through the entry channel 54 and positioned within the stromal pocket 42. FIG. 9 shows an example of a reshaped cornea 18′ that results after a biomechanical type corneal implant 36 is inserted into a stromal pocket 42.

In accordance with the methods of the present invention, a channel-shaped stromal pocket 60 can be established in the stroma 30 to accommodate a ring-shaped implant 36b, as can be seen by cross referencing FIGS. 10 and 11. Although the channel-shaped stromal pocket 60 is preferably located in the corneal periphery surrounding the central optical zone 62 of the cornea 18 as shown in FIGS. 10 and 11, it is to be appreciated that in accordance with the present invention, the channel-shaped stromal pocket 60 can be positioned anywhere within the stroma 30 of the cornea 18.

As shown in FIG. 11, the channel-shaped stromal pocket 60 can be formed with an anterior surface 64 and a posterior surface 66. With combined reference to FIGS. 10 and 11, it can be seen that the anterior surface 64 may be shaped as an annulus having a circular inner edge 68 and circular outer edge 70. Similarly, the posterior surface 66 may be shaped as an annulus having a circular inner edge 72 and a circular outer edge 74. As shown in FIG. 11, the channel-shaped stromal pocket 60 is further formed with an inner surface 76 and an outer surface 78. The inner surface 76 connects the inner edge 68 of the anterior surface 64 to the inner edge 72 of the posterior surface 66. Similarly, the outer surface 78 connects the outer edge 70 of the anterior surface 64 to the outer edge 74 of the posterior surface 66. The thickness 80 of the channel-shaped stromal pocket 60 is defined by the distance between the anterior surface 64 and the posterior surface 66. Although the channel-shaped stromal pocket 60 is shown in FIGS. 10 and 11 with the anterior surface 64 and the posterior surface 66 oriented normal to the visual axis 82, it is to be appreciated that in accordance with the present invention, the surfaces 64, 66, 76, 78 of the channel-shaped stromal pocket 60 can be oriented at any angle with respect to the visual axis 82. Further, although the surfaces 64, 66, 76, 78 of the channel-shaped stromal pocket 60 are shown in FIG. 11 as being substantially flat, it is to be appreciated that the surfaces 64, 66, 76, 78 can be rounded.

In accordance with the methods of the present invention, an entry channel 84 is created that is sized to allow the prescribed ring-shaped implant 36b to pass from an extracorporeal location, through the entry channel 84 and into the channel-shaped stromal pocket 60. As can be seen by cross-referencing FIGS. 10 and 11, an incision is made in the cornea 18 extending from the anterior surface 86 of the cornea 18 to the channel-shaped stromal pocket 60 to create the entry channel 84.

While the particular Method of Creating Stromal Pockets for Corneal Implants as herein shown and disclosed in detail is fully capable of obtaining the objects and providing the advantages herein before stated, it is to be understood that it is merely illustrative of the presently preferred embodiments of the invention and that no limitations are intended to the details of the construction or design herein shown other than as defined in the appended claims.

What is claimed is:

1. A method for creating a stromal pocket in a cornea for receiving a corneal implant, the cornea having a central optical zone surrounded by a corneal periphery, said method comprising the steps of:
directing the focal point of a pulsed laser beam to a point in the stroma of the cornea;
moving said focal point of said pulsed laser beam along a predetermined path in the stroma of the cornea to photodisrupt a preselected volume of stromal tissue, said preselected volume including at least a portion of the central optical zone of the cornea and being shaped for receipt of a corneal implant; and
incising an entry channel in the cornea, said entry channel extending from the anterior surface of the cornea to said preselected volume of stromal tissue.

2. A method as recited in claim 1 wherein said incising step is accomplished using a pulsed laser beam.

3. A method as recited in claim 1 further comprising the step of removing gas bubbles and debris from said preselected volume.

4. A method as recited in claim 1 wherein said preselected volume is substantially disk-shaped.

5. A method as recited in claim 1 wherein said preselected volume is lens-shaped having a first spherical surface and an opposed second spherical surface.

6. A method as recited in claim 1 wherein said preselected volume is located entirely with the central optical zone of the cornea.

7. A method as recited in claim 1 wherein said pulsed laser beam has a pulse duration of between approximately 500 picoseconds and approximately 10 femtoseconds.

8. A method as recited in claim 1 wherein the diameter of said focal point is between approximately 1 μm and approximately 100 μm.

9. A method as recited in claim 1 wherein said stromal pocket is formed at a preselected depth within the stroma.

10. A method for creating a stromal pocket in a cornea for receiving a corneal implant, the cornea having a central optical zone surrounded by a corneal periphery, said stromal pocket having an anterior surface, a posterior surface and a peripheral edge connecting said anterior surface to said posterior surface, said method comprising the steps of:
directing the focal point of a pulsed laser beam to a predetermined point in the stroma of the cornea;
moving said focal point of said pulsed laser beam along a predetermined path in the stroma of the cornea to photodisrupt stromal tissue to create said posterior surface, said anterior surface and said peripheral edge of said stromal pocket, at least a portion of said posterior surface passing through at least a portion of the central optical zone of the cornea; and
incising an entry channel in the cornea, said entry channel extending from the anterior surface of the cornea to said stromal pocket.

11. A method as recited in claim 10 further comprising the step of photodisrupting a volume of stromal tissue, said volume of stromal tissue being bounded by said anterior surface, said posterior surface and said peripheral edge.

12. A method as recited in claim 10 further comprising the step of passing a corneal implant through said entry channel and into said volume of stromal pocket.

13. A method as recited in claim 10 wherein said incising step is accomplished using a pulsed laser beam.

14. A method as recited in claim 10 further comprising the step of removing gas bubbles and debris from said stromal pocket.

15. A method as recited in claim 10 wherein said pulsed laser beam has a pulse duration of between approximately 500 picoseconds and approximately 10 femtoseconds.

16. A method as recited in claim 10 wherein said anterior surface and said posterior surface are created simultaneously.

17. A method as recited in claim 10 further comprising the step of excising a volume of stromal tissue from the cornea, said volume of stromal tissue being bounded by said anterior surface, said posterior surface and said peripheral edge.

18. A method as recited in claim 17 further comprising the step of passing a corneal implant through said entry channel and into said volume of stromal tissue.

19. A method as recited in claim 10 wherein the diameter of said focal point is between approximately 1 μm and approximately 100 μm.

20. A method as recited in claim 10 wherein said stromal pocket is formed at a preselected depth within the stroma.

21. A method for implanting a corneal implant into a cornea having a central optical zone surrounded by a corneal periphery, said method comprising the steps of:
directing the focal point of a pulsed laser beam to a predetermined point in the stroma of the cornea;
moving said focal point of said pulsed laser beam along a predetermined path in the stroma of the cornea to create a stromal pocket, said stromal pocket including at least a portion of the central optical zone of the cornea;
incising an entry channel in the cornea, said entry channel extending from the anterior surface of the cornea to said stromal pocket;
removing gas bubbles and debris from said stromal pocket; and
passing said corneal implant through said entry channel and into said stromal pocket.

22. A method as recited in claim 21 wherein said corneal implant is a biomechanical implant for altering the shape of the cornea.

23. A method as recited in claim 22 wherein said biomechanical implant is made from a material selected from the group consisting of polymeric materials, cellulose esters, hydrogel materials, silicone and bio-engineered tissue.

24. A method as recited in claim 21 wherein said corneal implant is an optical implant for altering the refractive properties of the cornea.

25. A method as recited in claim 24 wherein said optical implant is made from a material selected from the group consisting of polymeric materials, cellulose esters, hydrogel materials, silicone and bio-engineered tissue.

26. A method as recited in claim 21 wherein said corneal implant is lens-shaped having a first spherical surface and an opposed second spherical surface.

27. A method as recited in claim 21 wherein said stromal pocket is formed at a preselected depth within the stroma.
